SEQUENCE LISTING (11 sequences)

SEQ ID NO: 1 - DNA, 85 bp, Artificial Sequence
  aptamer
taatacgactcactatagcaatggtacggtacttccaagctaaccctcatctgcgcgctcccaaaagtgcacgctactttgctaa

SEQ ID NO: 2 - DNA, 23 bp, Artificial Sequence
  primer
gactcactatagcaatggtacgg

SEQ ID NO: 3 - DNA, 20 bp, Artificial Sequence
  primer
aaagtagcgtgcacttttgg

SEQ ID NO: 4 - RNA, 44 nt, Artificial Sequence
  aptamer
gccuguaagguggucgguguggcgaguguguuaggagagauugc

SEQ ID NO: 5 - RNA, 44 nt, Artificial Sequence; identical to SEQ ID NO: 4
  aptamer

SEQ ID NO: 6 - RNA, 44 nt, Artificial Sequence; identical to SEQ ID NO: 4
  aptamer

SEQ ID NO: 7 - RNA, 44 nt, Artificial Sequence; identical to SEQ ID NO: 4
  aptamer

SEQ ID NO: 8 - DNA, 46 bp, Artificial Sequence
  aptamer
acacgatgggggggtatgatttgatgtggttgttgcatgatcgtgg

SEQ ID NO: 9 - DNA, 72 bp, Artificial Sequence
  aptamer
ggcaggaagacaaacacgatgggggggtatgatttgatgtggttgttgcatgatcgtggtctgtggtgctgt

SEQ ID NO: 10 - DNA, 72 bp, Artificial Sequence
  aptamer
ggcaggaagacaaacacacaagcgggtgggtgtgtactattgcagtatctattctgtggtctgtggtgctgt

SEQ ID NO: 11 - DNA, 72 bp, Artificial Sequence; identical to SEQ ID NO: 10
  aptamer

(19) United States
(12) Patent Application Publication
(10) Pub. No.: US 2021/0140951 A1
(43) Pub. Date: May 13, 2021

(54) APTAMER BASED METHODS FOR PROTEIN DETECTION

(71) Applicant: QUANTUMDX GROUP LIMITED, Newcastle Upon Tyne, Tyne & Wear (GB)

(72) Inventors: Jonathan O'HALLORAN, Newcastle Upon Tyne, Tyne & Wear (GB); Stephen OSBORNE, Newcastle Upon Tyne, Tyne & Wear (GB); Henry STAINES, London, Greater London (GB)

(21) Appl. No.: 16/492,060

(22) PCT Filed: Mar. 6, 2018
(86) PCT No.: PCT/GB2018/050566

§ 371 Date: Sep. 6, 2019

(30) Foreign Application Priority Data
Mar. 7, 2017 (GB) 1703625.2

(51) Int. Cl. G01N 33/53; B01L 3/00; C12Q 1/6851; B01J 20/02; C12N 15/115; C12Q 1/6816

(52) U.S. Cl. CPC G01N 33/5308; B01L 3/502761; C12Q 1/6851; B01J 20/0251; G01N 2333/4737; C12Q 1/6816; C12N 2310/16; C12N 2310/3517; C12N 15/115

(57) ABSTRACT

The present invention relates to methods or assays for detecting proteins or molecules of interest in a sample. Aptamers specific to the protein of interest are obtained and are then incubated with the sample. If the protein of interest is present in the sample, protein-aptamer conjugates are formed. The incubated sample is then passed through a sorbent material designed to retain proteins and other cellular material whilst allowing free nucleic acid to flow through substantially unimpeded. If aptamer is detected in the output material at levels equivalent to the aptamer concentrations initially, this is indicative that no protein of interest was present in the sample, whereas if no (or potentially reduced) amounts of aptamer is detected in the flow through this is indicative of protein of interest in the sample as this will have bound to the aptamers and been retained by the sorbent material.

The present invention relates to methods or assays for detecting proteins or molecules of interest in a sample. Nucleic acid (DNA and RNA) aptamers specific to the protein of interest are obtained and are then incubated with the sample. If the protein of interest is present in the sample, protein-aptamer conjugates are formed. The incubated sample is then passed through a sorbent material designed to retain proteins and other cellular material whilst allowing free nucleic acid to flow through substantially unimpeded. If aptamer is detected in the output material at levels equivalent to the aptamer concentrations initially, this is indicative that no protein of interest was present in the sample, whereas if no (or potentially reduced) amounts of aptamer is detected in the flow through this is indicative of protein of interest in the sample as this will have bound to the aptamers and been retained by the sorbent material.

The rapid detection or identification of specific proteins in a sample can be of use in many different areas. For example, diagnostic testing for various diseases can be based on protein testing or a combination of protein and DNA and/or RNA testing.

One example of where detection of proteins is valuable is when looking at C-reactive protein (CRP), sometimes called an acute phase protein. A validated and clinically utilised indicator of bacterial infection is already based on CRP. The level of CRP increases when you have certain diseases that cause inflammation. CRP can be measured in a blood or serum sample. Normal concentration in healthy human serum is between 5 and 10 mg/L (increasing with aging). Higher levels are found in late pregnant women, mild inflammation and viral infections (10-40 mg/L), active inflammation, bacterial infection (40-200 mg/L), severe bacterial infections and burns (>200 mg/L). Examples of where the detection of CRP protein can be useful are;
Certain infections (mainly bacterial infections)
Abscesses
Rheumatoid arthritis
Various other muscular and connective tissue disorders—for example, polymyalgia rheumatica, giant cell arteritis or systemic lupus erythematosus
Tissue injury and burns
Some cancers—for example myeloma and Hodgkin's lymphoma
Crohn's disease
Rejection of an organ transplant
After operations

Currently, ELISA, immunoturbidimetry, nephelometry, rapid immunodiffusion, and visual agglutination are all methods used to measure CRP but it would be beneficial to provide a rapid, low cost test option that can be carried out at the point-of-care (POC).

It can be seen that it would be beneficial to provide a rapid protein detection assay. It would also be beneficial if such an assay could be carried out at the point of care.

According to the present invention, there is provided a method for detecting a target molecule in a sample, comprising;

obtaining an aptamer that will specifically bind to the target molecule of interest; incubating the sample with the aptamer;

passing the sample/aptamer mix through a material that is able to separate the target molecule of interest, from free nucleic acids e.g. aptamers by retaining or retarding the molecule of interest whilst allowing nucleic acids to pass through; detecting whether any aptamer is present in the output.

More preferably, there is provided a method for detecting a target molecule in a sample, comprising;

specific protein of interest in a sample, comprising;

obtaining an aptamer that will specifically bind to the protein of interest; incubating the sample with the aptamer;

passing the sample/aptamer mix through a material that is able to separate proteins from free nucleic acids/aptamers by retaining or retarding proteins whilst allowing nucleic acids e.g. aptamers to pass through;

detecting whether any aptamer is present in the output.

Unless reasonable to do so the steps of the method will be carried out in the order shown above.

Advantageously, the method allows rapid identification of specific target proteins or molecules in a sample, with a reduction of aptamer (or no aptamer) in the output being indicative of the presence of the protein or molecule in the sample.

Reference to free nucleic acids are to unbound nucleic acids, for example aptamers that are not bound to a protein or molecule.

Optionally the method can comprise pre-treatment steps such as lysis of cells in the sample.

Preferably the aptamer is selected to specifically bind the protein or molecule of interest in the sample type. For example, the aptamer may have been designed or selected to bind specifically, and with high affinity, to C-reactive protein in serum or blood.

Preferably the aptamer is designed or selected to have minimal cross-reactivity.

Preferably the aptamer has a high affinity constant. This usually means more and stronger binding is achieved quickly.

Preferably the aptamer is selected to be both highly specific for the target protein in the back ground of all the other proteins in the sample but also selected to not bind to the column sorbent material (i.e. the material that is able to separate proteins from free nucleic acids by retaining or retarding proteins whilst allowing nucleic acids to pass through), given the very high surface area for contact.

Preferably the step of detecting whether any aptamer is present in the output comprises;

amplifying aptamers present in the output and detecting whether any amplified aptamer is present.

Preferably the amplification of the aptamers consists of providing at least one primer pair specific for the aptamer, and a polymerase, and carrying out a PCR reaction where the aptamer, if present, is the template.

Alternatively, the aptamer is labelled, e.g. with a radio-nucleotide, and the label is detected, e.g. by performing a radio-graph following separation on a polyacrylamide gel. Similarly, a chemiluminescent tag could be used. Both of these options could be performed either directly or indirectly through hybridization to a complementary probe. Another alternative is that the aptamer could be put through a nanopore or sequenced/quantified using a next-generation sequencing device Optionally there is a dilution step after the incubation step.

Optionally a protein concentrate is flowed through the material in advance of or along with the sample/aptamer mix.

In some cases, the material performs better when a source of protein has been included.

Preferably the protein concentrate is bovine serum albumen (BSA).

It is preferred that the aptamer is titrated to provide the appropriate volume for the correct clinical diagnostic window.

Optionally, at least part of the method is carried out on a microfluidics chip or cartridge. Preferably the microfluidics chip or cartridge is associated with a point of care device.

Preferably the microfluidics chip or cartridge can also be used to detect DNA and/or RNA.

Preferably, the material that is able to separate proteins and/or molecules other than free nucleic acids from free nucleic acids by retaining or retarding proteins whilst allowing nucleic acids to pass through, is a sorbent material.

Optionally the sorbent material is able to separate proteins from free nucleic acids by retaining or retarding proteins whilst allowing nucleic acids to pass through and preferably comprises a substrate at least partially coated with benzyl methacrylate. Preferably said substrate comprises silica particles.

Whilst the preferred embodiment uses a sorbent material that is able to separate proteins from free nucleic acids by retaining or retarding proteins whilst allowing nucleic acids to pass through one skilled in the art could also utilise surfaces with attached antibodies, scFV's, other ligand binders or things like MIPs (molecular imprinted polymers).

According to another aspect of the present invention there is provided a kit for carrying out a protein detection assay, said kit comprising;

an aptamer that will specifically bind to the protein or molecule of interest; a material that is able to separate proteins from free nucleic acids by retaining or retarding proteins whilst allowing nucleic acids to pass through, in a form suitable for a sample to flow through;

primer pairs specific for the aptamer of interest.

Preferably the kit further comprises means for carrying out a PCR reaction.

Optionally, the kit comprises a microfluidics chip or cassette that comprises;

a means for receiving a sample;

at least one sorbent material chamber, downstream of the means for receiving the sample, containing said material that is able to separate proteins from free nucleic acids by retaining or retarding proteins whilst allowing nucleic acids to pass through;

at least one PCR chamber, downstream of the sorbent material chamber, adapted to allow a PCR reaction to occur.

According to another aspect of the present invention, there is a method for detecting C-reactive protein a sample, comprising;

obtaining an aptamer that will specifically bind to C-reactive protein; incubating the sample with the aptamer;

passing the sample/aptamer mix through a material that is able to separate proteins from free nucleic acids by retaining or retarding proteins whilst allowing nucleic acids to pass through in the output material;

carrying out a quantitative real-time polymerase chain reaction on the output material, using a pair of primers specific for the aptamer;

determining the presence of aptamer in the output.

Advantageously the method of detecting C-reactive protein a sample may be used as a method of diagnosing inflammation, in particular inflammation associated with bacterial infection.

Optionally, the method may include additional steps of testing a second sample from the same source to determine the effectiveness of antibiotic treatment.

According to another aspect of the present invention there is provided a method for detecting a specific protein or molecule of interest in a sample, comprising; obtaining a first aptamer that will specifically bind to the protein or molecule of interest;

incubating the sample with the aptamer;

passing the sample/first aptamer mix through a material that is able to separate proteins from free nucleic acids/aptamers by retaining or retarding proteins whilst allowing nucleic acids e.g. aptamers to pass through;

washing to remove any unbound or a specifically bound first aptamer;

obtaining a second aptamer with a higher binding affinity to the same binding site as the first aptamer that will specifically and competitively bind to the same protein or molecule of interest as the first aptamer;

passing the second aptamer through a material that is able to separate proteins from free nucleic acids/aptamers by retaining or retarding proteins whilst allowing nucleic acids e.g. aptamers to pass through such that the second aptamer will displace the first aptamer;

detecting whether any first aptamer is present in the output.

Unless reasonable to do so the steps of the method will be carried out in the order shown above.

Advantageously, as the use of the competing aptamer means that it is the presence of first aptamer that is indicative of

Preferably, when passing the second aptamer through a material that is able to separate proteins from free nucleic acids by retaining or retarding proteins whilst allowing nucleic acids to pass through the second aptamer is incubated with the material for a period of time before being displaced.

Advantageously this provides time for the competitive removal of first aptamer from the protein of interest by the second aptamer. By displacing a set amount of material after incubation it also allows a set volume of eluate to be used for downstream processing.

Preferably the first aptamer comprises flanking 3′ and 5′ primer sequences. In many cases the inclusion of flanking sequences will be sufficient to lower the binding affinity of the aptamer for the target when compared to an aptamer without said flanking sequences.

In order to provide a better understanding of the present invention we will hereby describe embodiments of the invention by way of example only. Such embodiments should not be considered unduly limiting as it would be understood that the invention can be utilised for the detection of a wide range of proteins and molecules by using the appropriate specific aptamers. The embodiments will be described with reference to the following figures in which;

FIG. 1 is a flow diagram showing the method (with optional dilution step) and the resulting reduction of aptamer which is indicative of the presence of a protein of interest; and

FIG. 2 shows a gel image of the results of the proof of concept work carried out with mouse IgG and using specific mouse IgG aptamers. From left to right, after the 100 bp marker lane, the lanes are (1) Mouse IgG+aptamer+BSA during column spin, (2) Mouse IgG+aptamer (no BSA), (3) PCR Negative control, (4) PCR positive control, (5) Goat IgG+aptamer+BSA during column spin, (6) aptamer alone+BSA during column spin, (7) Blank; and

FIG. 3 is a diagram showing a competitive version of the assay that can be utilised or is particularly useful when the target or protein/molecule of interest may be present in low concentration or copy number

Throughout this document the term ‘aptamer(s)’ or ‘aptamer sequences(s)’ refers to single-stranded nucleic acid molecules that show high-affinity binding to a target molecule such as a protein, polypeptide, lipid, glycoprotein, glycolipid, glycopeptide, saccharide, or polysaccharide. In certain preferred embodiments, the single-stranded nucleic acid is ssDNA, RNA or derivatives thereof. The aptamer comprises a three-dimensional structure held in certain conformation(s) that provide intermolecular contacts to specifically bind its given target. Although aptamers are nucleic acid based molecules, the binding to the target molecule is not entirely dependent on a linear base sequence, but rather a particular secondary/tertiary/quaternary structure. The term also covers next generation aptamers such as X aptamers that can't be typically amplified by PCR but can be adapted by adding a link primer. Such aptamers can specifically bind to proteins of interest but can also be easily amplified, sequenced etc. in a downstream process. The term also covers aptamers that include modified bases. It is envisaged that the aptamers may include traditional aptamers of 15 to 120 bases in length, as well as longer aptamers of approx. 200 bases in length (e.g. (e.g., Ultramers® by Integrated DNA Technologies, Inc. Coralville, Iowa, USA).

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C-Ci4aryl group, a C6-Cio aryl group, or a C6aryl group. Examples of aryl groups include, but are not limited to, benzene, naphthalene and azulene. An aryl group may be unsubstituted or substituted, e.g., substituted with methyl or methoxy, and the connection of the aryl group to other parts of a larger molecule may be via the aryl ring or via the substituent. Examples of aryl groups that are substituted include benzyl, hydroxybenzyl, 2-phenylethyl, benzhydryl, triphenylmethyl, anisolemethyl, phenylethanol, and naphthalenemethyl.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted. e.g., substituted with methyl or methoxy, and the connection of the heteroaryl group to other parts of a larger molecule may be via the heteroaryl ring or the substituent. Pyridinemethyl and thiophen-3-ylmethyl are examples of heteroaryl groups that are substituted.

The general methodology is shown in FIG. 1. A sample, either containing protein of interest (left column) or not containing protein of interest (right column), is incubated with an amount of aptamer, the aptamer being specific for the protein of interest. The amount of aptamer is at least the amount required to be robustly detectable by end-point PCR. During incubation, the aptamer will bind to the protein of interest in the sample where it is present to produce protein-aptamer conjugates, but will remain unbound in the control sample where no protein of interest is present.

In many cases, the apatamers will be commercially available, however it is known in the art that aptamers can be produced using an exponential enrichment (SELEX) method to acquire aptamers against a target. Typically, the aptamer can be optimised for binding in a particular sample type i.e. serum.

Advantages of using aptamers include that they don't require fabrication in cells or animals, which results in them being cost effective to manufacture; they exhibit minimal differences between batches; they are not easily influenced by environmental factors such as external temperature, humidity, and the like, and as such can be stored easily.

After incubating for an appropriate amount of time e.g. 30 minutes at 37° C., a dilution or buffer exchange step can optionally be carried out. This ensures that the sample is in a buffer that is appropriate for further processing i.e. it is appropriate for PCR etc.

The incubated sample is then flowed through or over a protein retention column that contains material that is able to retain and/or substantially retard proteins whilst allowing nucleic acids to pass through relatively unimpeded. In a preferred embodiment the protein retention column comprises a spin column packed with beads, the beads being a silica material at least partially coated with benzyl methacrylate. Such beads can be made by suspending silica in a solution of dimethylvinylchlorosilane in trifluorotoluene; removing the liquid and resuspending the silica in a fresh solution of dimethyivinylchliorosilane in trifluorotoluene; optionally removing the liquid and resuspending the silica again in a fresh 5% solution of dimethylvinylchlorosilane in trifluorotoluene; collecting and drying the resulting silanized silica; adding the silanized silica, benzylmethacrylate and potassium peroxodisulfate to a stirred solution of sodium stearate in water; and collecting and drying the resultant sorbent material comprising silanized silica coated with poly(benzyl methacrylate). Exemplary materials that are able to retain and/or substantially retard proteins whilst allowing nucleic acids to pass through relatively unimpeded are described in WO2016/040697.

As is taught by WO2016/040697 the sorbent material which will retain or substantially retard proteins whilst allowing nucleic acids to pass through relatively unimpeded may in a preferred embodiment comprise a silanized material at least partially coated or formed with a polymer selected from the group consisting of a poly(aryl methacrylate), a poly(aryl acrylate), a poly(heteroaryl methacrylate, a poly(heteroaryl acrylate) and a copolymer thereof. In one variation, the silanized inorganic material is selected from the group consisting of a silanized silica particle, a silanized silica fiber and a silanized silica membrane but could also be a porous organic material or membranes. In another variation, the polymer can include a recurring unit selected from the group consisting of anisolemethyl methacrylate, phenylethanol methacrylate, pyridinemethyl methacrylate and naphthalenemethyl methacrylate.

Alternative materials could also be used if they are able to substantially retain or retard proteins whilst allowing nucleic acids to pass through relatively unimpeded. Examples may include material described in WO/2005/095476. This describes the use of a polymer obtainable by a process of polymerizing at least three components [A], [B], and [M] wherein

[A] is at least one aromatic or aliphatic compound having at least one polymerisable unsaturated moiety,

[B] is at least one cross-linkable aromatic or aliphatic compound, and

[M] is an organic non-saturated polymerisable compound different from [A] having hetero atoms in the C—C chain or in side chains wherein the process comprises the steps of
admixing the components [A], [B], and [M] sequentially or non-sequentially,
polymerising the resulting mixture composition
removing unreacted material and
recovering and drying the composite material.

A particular polymer that could be used in the invention as a sorbent material comprises the following structure

{[A]ₓ-[B]_{y}-[M]_{z}}ₚ

wherein [A], [B], and [M] have the same meaning as above and x, y, and z are independent of each other, an integer of 1-100 and p is a number between 2 and 5000. Also envisaged is a monomer having the structure [A]ₓ-[B]_{y}-[M]_{z} wherein [A], [B], and [M] have the same meaning as above and x, y, and z is independent of each other, an integer of 1-100

If the sample contained the protein of interest, it will now contain protein-aptamer conjugates and at least a portion of the aptamer, and in some cases substantially all of the aptamer, will be bound to protein. As the protein is retained in the column, the aptamer is also retained and will not be present in the output from the column. If the sample does not contain the protein of interest, the aptamer will pass through the column freely and will be present, in substantially the same amount, in the output from the column. A significant reduction or absence of aptamer in the output is therefore indicative of the presence of the protein of interest. The aptamer can be detected by providing a primer pair specific to the aptamer and carrying out a polymerase chain reaction (PCR) to amplify the amount of aptamer present detection.

Proof of Concept Using Mouse IgG and Mouse IgG Specific Aptamers

In order to prove the methodology, mouse IgG protein was obtained along with a mouse IgG specific aptamer that specifically binds to said mouse IgG protein. The mouse IgG specific aptamer (SEQ ID 1) had the following sequence;

TAATACGACTCACTATAGCAATGGTACGGTACTTCCAAGCTAACCCTCAT
CTGCGCGCTCCCAAAAGTGCACGCTACTTTGCTAA

The aptamer was diluted in DNase free H₂O. 100 uM Stock serially diluted in H2O 9×, by a factor of 10 each dilution to a working stock of 100 femtomolar.

As a control, goat IgG protein was also obtained (it was anticipated that the mouse IgG specific aptamer would not bind to this protein).

Mouse IgG specific aptamer was incubated alone, or in the presence of excess mouse IgG, or goat IgG to provide test sample. In some cases, bovine serum albumin (BSA) was also included. The incubation was for 30 minutes at 37° C.

10 μl of test sample was added to 70 μl of phosphate-buffer saline (PBS) as a dilution step and to simulate a typical lysis buffer step that would be used for blood or serum samples.

5% BSA was included in some test samples to provide additional protein volume that may, in some circumstances, improve the overall protein retention by the column of sorbent material. This could be provided as a pre-wash step or included with the sample if it is used.

Sorbent Columns (DNA-XT™ columns) were obtained from QuantuMDx Group Limited. The sorbent columns comprise a sorbent material, in this case silica particles coated with benzyl methacrylate, packed into a spin column. The particles are 15 μm in diameter. 50 mg of sorbent material is packed into 1 ml-capacity spin columns, which are in turn placed in collection tubes. Particles are sealed in the column using 7 mm-diameter polyethylene frits (hydrophilic).

40 μl of test sample was applied to wetted DNA-XT™ columns and incubated for 3 min prior to a 1 minute spin at 5000 rpm. [symbol] The output material (i.e. the material that has passed through the column without being substantially retained or retarded by the sorbent material) was then collected.

8 μl of flow through material, to account for 1/8 dilution of original sample, was used in a PCR reaction (35 cycles). The PCR reaction was carried out in accordance with known methods as would be well understood by one skilled in the art. The following forward and reverse primers were used in the PCR reaction, as they are specific for the mouse IgG specific aptamer;

Primer_F
(SEQ ID No 2)
GACTCACTATAGCAATGGTACGG
Primer_R
(SEQ ID No 3)
AAAGTAGCGTGCACTTTTGG

20 μl of each 50 μl PCR reaction was run on a TBE/2% agarose gel along with negative and positive PCR controls. The results are shown in FIG. 2.

Detection of CRP to Differentiate Between Bacterial and Viral Infections

A particular example where the method can be used is in relation to detecting C-reactive protein (CRP) in a serum or blood sample in order to determine whether an infection is viral or bacterial. This method could also be used to check or monitor the effectiveness of an antibiotic.

Many papers reference CRP noting its use for cardiovascular issues, however it is also of value in differentiating between bacterial and viral infections. This is particular importance given the rising prevalence of antibiotic resistance and the strong correlation between antibiotic resistance and overuse of antibiotics. It is thought that over 80% of antibiotics prescribed in primary care are unnecessary and a rapid and simple screening test that would ascertain whether an infection had a bacterial or viral basis would be of significant benefit.

CRP is an acute-phase protein synthesised in the liver. CRP is normally present in very low concentrations in the blood of healthy individuals. In bacterial infections, CRP concentrations markedly increase whilst in viral infections result in no or very little increase in CRP levels. For example, the CRP range in the blood of a normal healthy individual is 1-3 mg/l, this rises to 150-350 mg/l in cases of invasive bacterial meningitis and greater than 500 mg/l in high risk cases such as sepsis [Jaye and Waites 1997 via Pultar 2009]. More generally 99% of people have CRP levels of les than 10 mg/l in their blood and as such this can generally be considered as a cut off for inflammatory disease based on bacterial infection, with higher levels indicative of inflammatory disease.

In an embodiment of the present invention, a test could use aptamers that are specific for CRP. For example, the following aptamers are already known and could be used;

| Paper | Aptamer Sequence |
|---|---|
| Pultar | 5-GCC UGU AAG GUG GUC GGU GUG GCG AGU |
| 2009 | GUG UUA GGA GAG AUU GC-3 (RNA) |
| Wang | 5′-Cy3-GCC UGU AAG GUG GUC GGU GUG GCG |
| 2011 | AGU GUG UUA GGA GAG AUU GC-3′ |
| | 5′-GCC UGU AAG GUG GUC GGU GUG GCG AGU |
| | GUG UUA GGA GAG AUU GC-Cy5-3′ |
| | 5′-biotin-GCC UGU AAG GUG GUC GGU GUG |
| | GCG AGU GUG UUA GGA GAG AUU GC-3′ |
| | (RNA) |
| Wang | 5′-ACA CGA TGG GGG GGT ATG ATT TGA TGT |
| 2011 | GGT TGT TGC ATG ATC GTG G-3′ |
| | (DNA) |
| Huang | 5′-GGCAGGAAGACAAACACGATGGGGGGGTATGATTT- |
| 2010 | GATGTGGTTGTTGCATGATCGTGGTCTGTGGTGCTGT-3′ |
| | (DNA) |
| Hwang | 5′-Biotin- |
| 2016 | GGCAGGAAGACAAACACACAAGCGGGTGGGTGTGTACTAT |
| | TGCAGTATCTA TTCTGTGGTCTGTGGTGCTGT-FAM-3 |
| | 5′-Biotin-GGCAGGAAGACAAACACACAAGCGGG |
| | TGGGTGTGTACTATTGCAGTATCTATTCTGTGGTCTGTGG |
| | TGCTGT-3′ |
| | (DNA) |

It will be appreciated that aptamer selection will be based on the conditions in which the test will occur, and the above list is exemplary rather than limiting. As an alternative, specific aptamers could be created or selected for using known techniques, for example SELEX methodology could be used.

The selected aptamer(s) are mixed or incubated with a blood or serum sample for a period of time, e.g. 15 minutes, and then passed through a protein retention column that contains material that is able to retain or substantially retard proteins. This could for example be a DNA-XT™ column as identified previously, which utilises silica particles coated with benzyl methacrylate to retain protein whist allowing nucleic acid material to pass through as an output material.

A Quantitative real-time PCR (qRT-PCR) reaction is then carried out using the output material. The primers that are used in the PCR reaction are designed to be specific for the aptamer such that if aptamer is present it will be amplified.

The results can be used to determine the presence of a bacterial infection with significant volumes of aptamer being indicative that no, or low levels, of CRP is present, which in turn indicates no source of bacterial infection.

Detection of Other Proteins/Molecules

Other potential protein targets for which the invention may be useful are;

Myxovirus resistance protein A (MxA)

Acute phase reactants:

Orosomucoid

Procalcitonin

Innate Immune responses:

Interferon-gamma

Interferon-likeproteins

Acquired responses:

Immunoglobulins

Surface proteins expressed on T/B-cells

This list should not be considered as being limiting.

Tuning of Detection

There are several options to further tune the methods of the invention. Aptamer concentration, primer concentrations, PCR conditions, incubation time/conditions. Typically, the limit of detection conditions will be determined for a given target and then “tuning” will be carried out to ensure the assay sensitivity is sufficient to measure to an appropriate threshold concentration of interest (for a qualitative result).

Low Copy Number Target Detection

One particular option that is envisaged to utilize the invention for lower copy number or low concentration targets is to provide an additional competitor aptamer into the methodology. In cases where the protein or molecule of interest is potentially present only in low concentrations or copy numbers it can be challenging to identify using the basic methodology as the reduction in aptamer present in the output may show only a relatively small reduction. In this case a variation on the standard methodology can be employed which uses two aptamers to the same binding area, one having a higher affinity than the other such that it will successfully compete for the binding site.

The basic steps for this method are to create or obtain two aptamers to the same binding area on the target (e.g. protein or molecule of interest); the second aptamer having a higher affinity to said binding area than the first aptamer. In many cases the addition of the two primer sequences to the ends of the first aptamer will achieve the reduction in affinity without requirement for further modification whilst also allowing for downstream amplification—however one skilled in the art would understand how to create or obtain the first and second aptamers and that the modification and/or substitution of one or more bases within the first aptamer sequence may also be employed to lower its affinity as compared to the second aptamer. The aptamers are selected such that the second aptamer will preferentially bind to the site and will successfully compete with the first aptamer removing said first aptamer from said binding site when both aptamers are present. The aptamers are also selected such that they will not non-specifically bind to the material that the sample is flowed through.

An example method is shown below and is generally depicted in FIG. 3.

Exemplary Assay for a low copy protein of interest;
1. Incubate first, lower affinity, aptamer 2 with sample (said sample containing the protein of interest 1). The first aptamer 2 in this embodiment comprising 5′ and 3′ primer sequences 3a, 3b.
2. Run through a column containing material that is able to separate proteins from free nucleic acids by retaining or retarding proteins and allowing nucleic acids to pass through (e.g. DNA-XT™, a substrate coated with benzylmethacrylate) the column (depending on Kon/off)
3. Protein and bound first low affinity aptamer bind to column, excess first low affinity aptamer flows through.
4. Wash column (X times, preferably 3 times), to remove a specifically bound or unbound first low affinity aptamer.
5. Flow the second competitor, higher-affinity, aptamer 4 through the column i.e. the ‘competition phase’. In preferred embodiments one void volume's worth (i.e. a volume substantially equivalent to the volume of the interstitial space within the column) of the second competitor aptamer 4 is flowed on to the column and is allowed to incubate (the incubation will be under optimised conditions and the time selected to be appropriate for the aptamers and targets involved). Once the incubation is complete the 1 void volumes worth of liquid is displaced which keeps the volume to analyse downstream, e.g. by PCR, to a minimum. During this ‘competition phase’ the second higher affinity aptamer 4 will displace the first lower affinity aptamer 1 for binding to the protein of interest leaving the lower affinity (with comp. primer sequences) in the eluate. If the first lower affinity aptamer is then present in the output eluate this is indicative of the presence of the target protein of interest.
6. For downstream processing it is possible to add PCR mix with primers to the eluate and perform PCR which would increase the amount of detectable first aptamer.

Advantageously, by using this method variation where an additional competitive aptamer to the same binding site is used, the presence of protein (or other molecule) of interest is now identified by the presence of the first, lower binding affinity, aptamer in the final output as it will only be present if it had bound to the protein of interest and then been displace by the second aptamer. Further amplification of the first aptamer, if it is present in the output, can also be carried out such that even very low copy numbers could be detected. A parallel control could also be included where it is known no target was present to ensure the wash steps remove unbound or non-specifically bound first aptamer prior to the second aptamer being introduced.

It will be appreciated that features from one embodiment may be appropriately incorporated into another embodiment unless technically unfeasible to do so.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method for detecting a target molecule in a sample, comprising;
obtaining an aptamer that will specifically bind to the target molecule of interest;
incubating the sample with the aptamer;
passing the sample/aptamer mix through a material that is able to separate the target molecule of interest, from free nucleic acids e.g. aptamers by retaining or retarding the molecule of interest whilst allowing nucleic acids to pass through;
detecting whether any aptamer is present in the output.

2. A method as in claim 1 for detecting a target protein in a sample, comprising
obtaining an aptamer that will specifically bind to the protein of interest;
incubating the sample with the aptamer;
passing the sample/aptamer mix through a material that is able to separate proteins from free nucleic acids by retaining or retarding proteins whilst allowing free nucleic acids to pass through;
detecting whether any aptamer is present in the output.

3. A method as in claim 2 wherein the aptamer is selected to specifically bind the protein or molecule of interest in the sample type.

4. A method as in any of the previous claims wherein the aptamer is designed or selected to have minimal cross-reactivity.

5. A method as in any of the previous claims wherein the step of detecting whether any aptamer is present in the output comprises;
amplifying aptamers present in the output and detecting whether any amplified aptamer is present.

6. A method as in any of the previous claims wherein the amplification of the aptamers consists of providing at least one primer pair specific for the aptamer, and a polymerase, and carrying out a PCR reaction where the aptamer, if present, is the template.

7. A method as in any of the previous claims wherein there is a dilution step after the incubation step.

8. A method as in any of the previous claims wherein a protein concentrate is flowed through the material in advance of, or along with, the sample/aptamer mix.

9. A method as in claim 8 wherein the protein concentrate is bovine serum albumen (BSA).

10. A method as in any of the previous claims wherein at least part of the method is carried out on a microfluidics chip or cartridge.

11. A method as in claim 10 wherein the microfluidics chip or cartridge is associated with a point of care device.

12. A method as in any of claim 10 or 11 wherein the microfluidics chip or cartridge can also be used to detect DNA and/or RNA.

13. A method as in any of the previous claims wherein the material that is able to separate proteins from free nucleic acids by retaining or retarding proteins whilst allowing nucleic acids to pass through, is a sorbent material.

14. A method as in claim 13 wherein the sorbent material comprises a substrate at least partially coated with benzyl methacrylate. Preferably said substrate comprises silica particles.

15. A kit for carrying out a protein detection assay, said kit comprising;
a first aptamer that will specifically bind to the protein or molecule of interest;
a material that is able to separate proteins from free nucleic acids by retaining or retarding proteins whilst allowing nucleic acids (including unbound aptamers) to pass through, in a form suitable for a sample to flow through;
primer pairs specific for the aptamer of interest.

16. A kit as in claim 15, further comprising means for carrying out a PCR reaction.

17. A kit as in claim 15 or 16, further comprising a second aptamer that is able to bind to the same target as the first aptamer but with higher binding affinity than said first aptamer.

18. A kit as in claims 15 to 17 wherein the kit comprises a microfluidics chip or cassette that comprises;
a means for receiving a sample;
at least one sorbent material chamber, downstream of the means for receiving the sample, containing said material that is able to separate proteins from free nucleic acids by retaining or retarding proteins whilst allowing nucleic acids to pass through;
at least one PCR chamber, downstream of the sorbent material chamber, adapted to allow a PCR reaction to occur.

19. A method for detecting C-reactive protein a sample, comprising;
obtaining an aptamer that will specifically bind to C-reactive protein;
incubating the sample with the aptamer;
passing the sample/aptamer mix through a material that is able to separate proteins from free nucleic acids by retaining or retarding proteins whilst allowing nucleic acids to pass through in the output material;
carrying out a quantitative real-time polymerase chain reaction on the output material, using a pair of primers specific for the aptamer;
determining the presence of aptamer in the output.

20. Use of the method of claim 19 for diagnosing inflammation, in particular inflammation associated with bacterial infection.

21. A method for determining the effectiveness of antibiotic treatment using the steps of testing a first sample obtained from a source for C-reactive protein using the method of claim 19; testing a second sample from the same source after treatment with an antibiotic and comparing the results.

22. A method for detecting a specific protein or molecule of interest in a sample, comprising;
obtaining a first aptamer that will specifically bind to the protein or molecule of interest;
incubating the sample with the aptamer;
passing the sample/first aptamer mix through a material that is able to separate proteins and/or molecules other than free nucleic acids from free nucleic acids by retaining or retarding proteins whilst allowing nucleic acids to pass through;
washing to remove any unbound or a specifically bound first aptamer;
obtaining a second aptamer with a higher binding affinity to the same binding site as the first aptamer that will specifically and competitively bind to the same protein or molecule of interest as the first aptamer;
passing the second aptamer through a material that is able to separate proteins and/or molecules other than free nucleic acids from free nucleic acids by retaining or retarding proteins whilst allowing nucleic acids to pass through such that the second aptamer will displace the first aptamer;
detecting whether any first aptamer is present in the output.

23. A method as in claim 22, wherein when passing the second aptamer through a material that is able to separate proteins from free nucleic acids by retaining or retarding proteins whilst allowing nucleic acids to pass through the second aptamer is incubated with the material for a period of time before being displaced.

24. A method as in claim 22 or 23 wherein the first aptamer contains flanking primer sequences.
